(19) United States
(12) Patent Application Publication
(10) Pub. No.: US 2018/0296092 A1
(43) Pub. Date: Oct. 18, 2018

(54) SYSTEM AND METHOD FOR MONITORING AND DETERMINING A MEDICAL CONDITION OF A USER

(71) Applicant: Healthymize Ltd, Julis (IL)

(72) Inventors: Shadi HASSAN, Julis (IL); Daniel ARONOVICH, Kibbutz Hannaton (IL)

(73) Assignee: Healthymize Ltd, Julis (IL)

(21) Appl. No.: 15/769,072

(22) PCT Filed: Oct. 19, 2016
(86) PCT No.: PCT/IL2016/051132

§ 371 Date: Apr. 18, 2018

Related U.S. Application Data
(60) Provisional application No. 62/243,680, filed on Oct. 20, 2015.
(60) Provisional application No. 62/252,587, filed on Nov. 9, 2015.
(60) Provisional application No. 62/274,598, filed on Jan. 4, 2016.

(51) Int. Cl. A61B 5/00; A61B 5/024; A61B 5/08

(52) U.S. Cl. CPC A61B 5/0022; A61B 5/02438; G08B 21/0205; A61B 5/0823; A61B 5/4842; A61B 5/4803

(57) ABSTRACT

A system and method for monitoring and determining a medical condition of a user, via a communication device of the user is disclosed. The system and a method according to some embodiments, may comprise a memory and a processor, the processor may be configured to receive an audio signal related to a user's speech, and determine a progress of a disease of the user based on comparing the audio signal to a reference audio signal. According to some embodiments, the memory and processor may be included in a mobile communication device operated by the user, the reference audio signal may be generated based on recording normal speech of the user e.g. during telephone calls.

### FIELD OF THE INVENTION

The present invention relates generally to home tele-monitoring systems and methods for monitoring and determining a medical condition of a user and/or patient. More specifically, the present invention relates to monitoring and determining a presence, state or progress of a disease based on speech analysis.

### BACKGROUND OF THE INVENTION

Lung Diseases (LD), Chronic obstructive pulmonary disease (COPD), Asthma, Chronic Heart Failure (CHF) and Chronic Kidney Disease (CKD) are known as a major public health problem worldwide. Known systems and methods include a simultaneous, multi-parameter physiological monitoring device, with local and remote analytical capability. A handheld medical diagnostic instrument that provides high time-resolution pulse waveforms associated with multiple parameters including blood pressure measurements, blood oxygen saturation levels, electrocardiograph (ECG) measurements, and temperature measurements is known in the art. Data from a handheld device can be analyzed onboard, with local computerized devices, and with remote server based systems. The remote server may be configured to analyze this data according to various algorithms chosen by the physician to be most appropriate to the patient's particular medical condition (e.g. COPD patient algorithms). The server may be further configured to automatically provide alerts and drug recommendations.

A known in the art automated system for monitoring respiratory diseases, such as asthma, provides noninvasive, multimodal monitoring of respiratory signs and symptoms that can include wheeze and cough. Some systems employ a mobile device, such as a cell phone, in which raw data from a microphone and an accelerometer are processed, analyzed, and stored. Analyses of a user's symptoms and activity level prior to, during, and after an event can provide meaningful determinations of disease severity and predict future respiratory events. The system can provide a summary of data, as well as an alarm when symptom severity reaches a threshold.

Yet another known system and method for monitoring a person suffering from a chronic medical condition predicts and assesses physiological changes which could affect the care of that subject. Monitoring includes measurements of respiratory movements, which can then be analyzed for evidence of changes in respiratory rate, or for events such as hypopneas, apneas and periodic breathing. Monitoring may be augmented by the measurement of nocturnal heart rate in conjunction with respiratory monitoring. Additional physiological measurements can also be taken such as subjective symptom data, blood pressure, blood oxygen levels, and various molecular markers.

Known computerized methods and systems for measuring a user's lung capacity and stamina include an application on a user's mobile communication device. For example, the application instructs the user to fill his lungs with air and utter vocal sounds while exhaling. The application measures the length of the vocal sounds receiving time. The time length is used to calculate user's lungs volumes. Such an application requires active participation from the patient and depends on the patient adherence.

There is a long felt unmet need in the art for an effective home monitoring system and method that doesn't require daily active participation from the patient and that further enable monitoring and determining a medical condition of a user.

### SUMMARY OF THE INVENTION

Embodiments of the present invention provide a system and method for monitoring and determining a medical condition of a user, via a communication device of the user. A system and a method according to some embodiments, may comprise a memory and a processor, the processor may be configured to: receive an audio signal related to a user's speech; and determine a progress of a disease of the user based on comparing the audio signal to a reference audio signal. According to some embodiments, the memory and processor may be included in a mobile communication device operated by the user, such as, for example, a laptop, a computer, a tablet, a kiosk, a smart phone, a telephone, a smart watch, a wearable device and a medical device.

According to some embodiments, the reference audio signal may be generated based on recording normal speech of the user.

According to some embodiments, the processor may be further configured to generate the reference audio signal by periodically recording speech of the user during telephone calls.

According to some embodiments, the processor may be further configured to obtain the audio signal by: presenting text to the user; instructing the user to read the text; and recording the user's speech.

According to some embodiments, the processor may be further configured to communicate the audio signal to a server and the server is configured to: determine a progress of a disease of the user based on comparing the audio signal to a reference audio signal; and based on the progress, send a message to a predefined recipient list.

According to some embodiments, the processor may be further configured to alert the user based on the determined medical condition.

According to some embodiments, determining a progress of the disease may include:

identifying, based on comparing the audio signal to a reference audio signal that a threshold is breached.

According to some embodiments, the threshold may be defined based on at least one user associated parameter, such as, for example, at least one user associated parameter selected from a group consisting: a location of the user, activity level of the user, medical history of the user, and recent hospitalization information of the user.

According to some embodiments, the threshold is defined based on an activity of the user, wherein identifying an activity of the user is based on input received from a component included in the system.

According to some embodiments, a system and method according to the present invention may include determining whether or not the user performed a physical activity prior to the determining of the progress of a disease; and if the user performed a physical activity prior to the determining of the progress of a disease then instructing the user to rest, obtaining an audio signal related to a user's speech, and determining a progress of a disease of the user based on comparing the audio signal to the reference audio signal.

According to some embodiments, the processor may be further configured to: if a difference between the recorded audio signal of the user and a baseline audio signal is greater than a threshold then: instructing the user to speak; recording the user's speech; and determining a progress of the disease based on comparing the recorded speech to the baseline audio signal.

According to some embodiments, the threshold may be personalized to each user based on outlier detection.

According to some embodiments, the processor may be further configured to: determine nonadherence with a prescribed treatment based on at least one of: comparing the audio signal to a reference audio signal and a report from an adherence system; and modifying the threshold according to a rule related to the user's medical condition and to the prescribed treatment.

According to some embodiments, the processor may be further configured to calculate a biomarker score for the user comparing the audio signal to a reference audio signal.

According to some embodiments, the processor may be further configured to record amount of speech per time unit over a predefined time interval and classify the user based on the amount of speech.

### BRIEF DESCRIPTION OF THE DRAWINGS

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale.

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1A shows high level block diagram of an exemplary computing device according to illustrative embodiments of the present invention;

FIG. 1B shows a user using an exemplary device according to illustrative embodiments of the present invention;

FIG. 2 is an overview of a system according to illustrative embodiments of the present invention; and

FIG. 3 shows a flowchart of a method according to illustrative embodiments of the present invention; and

FIG. 4 shows a flowchart of a method according to illustrative embodiments of the present invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

As described, known systems and methods require a patient to actively participate in a process of monitoring and determining the medical condition of the patient and therefore depend on the patient adherence. For example, in order to obtain a measurement of a condition of a patient, known systems and methods typically require an action to be performed by a physician or by the patient, other systems and methods require a dedicated device to be attached to a patient. Therefore, known systems and methods do not enable continuous and/or early detection related to, for example, LD, COPD, CHF and/or CKD, and other diseases that affect speech such as depression, neurological diseases, psychiatric diseases and/or other diseases, without interfering with the patient's activities and/or otherwise burdening the patient.

In contrast and as described, embodiments of the invention include a home monitoring system and method that doesn't require daily or other active participation of the patient. For example, embodiments of the invention enable automated, non-invasive characterization of LD, CHF and CKD severity, monitoring of LD, CHF and CKD status over time, and early recognition of a LD, CHF and CKD ‘flare-up’ for prompt institution of therapy where the characterization, monitoring and/or recognition are performed without requiring the patient to perform a specific activity. Moreover, in some embodiments, a process of monitoring, characterization and/or determination of a medical condition of a user may be performed without the user being aware of the process.

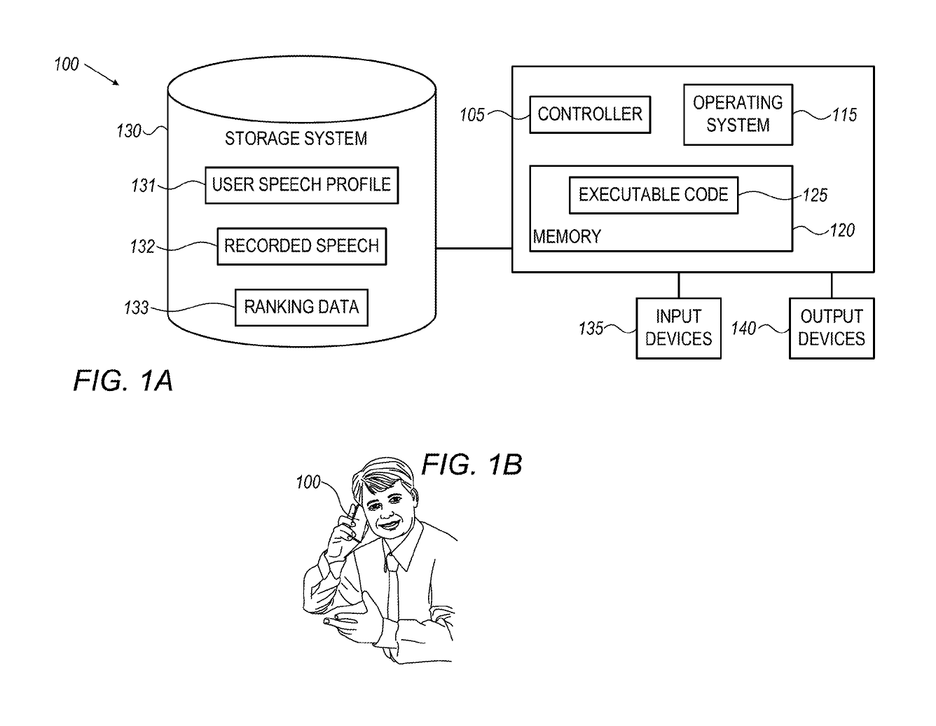

Reference is made to FIG. 1A, showing a high level block diagram of an exemplary computing device according to some embodiments of the present invention. In some embodiments, computing device 100 may be, or may be included in, a cellular telephone (e.g., smartphone or mobile phone as known in the art). Computing device 100 may include a controller 105 that may be, for example, a central processing unit processor (CPU), a chip or any suitable computing or computational device, an operating system (OS) 115, a memory 120, executable code 125, a storage system 130, input devices 135 and output devices 140. As shown, storage system 130 may include a user speech profile 131, recorded speech (or recorded audio signals) 132 and ranking data 133.

Controller 105 (or one or more controllers or processors, possibly across multiple units or devices) may be configured to carry out methods described herein, and/or to execute or act as the various modules, units, etc. More than one computing device 100 may be included in, and one or more computing devices 100 may be, or act as the components of, a system according to some embodiments of the invention.

OS 115 may be or may include any code segment (e.g., one similar to executable code 125 described herein) designed and/or configured to perform tasks involving coordination, scheduling, arbitration, supervising, controlling or otherwise managing operation of computing device 100, for example, scheduling execution of software programs or enabling software programs or other modules or units to communicate. OS 115 may be a commercial OS, e.g., OS 115 may be an Android or an iOS operating systems as known in the art.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of, possibly different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of OS 115. For example, executable code 125 may be an application that receives an audio signal related to a user's speech (or audio signal) and determines a presence, state or progress of a disease of the user based on relating or comparing the audio signal to a reference audio signal as further described herein. Although, for the sake of clarity, a single item of executable code 125 is shown in FIG. 1A, a system according to some embodiments of the invention may include a plurality of executable code segments similar to executable code 125 that may be loaded into memory 120 and cause controller 105 to carry out methods described herein.

Storage system 130 may be or may include, for example, a flash memory, a universal serial bus (USB) device or other suitable removable and/or fixed storage unit. Content may be stored in storage system 130 and may be loaded from storage system 130 into memory 120 where it may be processed by controller 105. In some embodiments, some of the components shown in FIG. 1A may be omitted. For example, memory 120 may be a non-volatile memory (e.g., a flash memory in a smartphone as known in the art) having the storage capacity of storage system 130. Accordingly, although shown as a separate component, storage system 130 may be embedded or included in memory 120.

Input devices 135 may be or may include a microphone, a mouse, a keyboard, a touch screen or pad or any suitable input device. It will be recognized that any suitable number of input devices may be operatively connected to computing device 100 as shown by block 135. Output devices 140 may include one or more displays or monitors, speakers and/or any other suitable output devices. It will be recognized that any suitable number of output devices may be operatively connected to computing device 100 as shown by block 140. Any applicable input/output (I/O) devices may be connected to computing device 100 as shown by blocks 135 and 140. For example, a wired or wireless network interface card (NIC) or unit, a printer, a universal serial bus (USB) device or an external hard drive may be included in, or connected to computing device 100 as, input devices 135 and/or output devices 140.

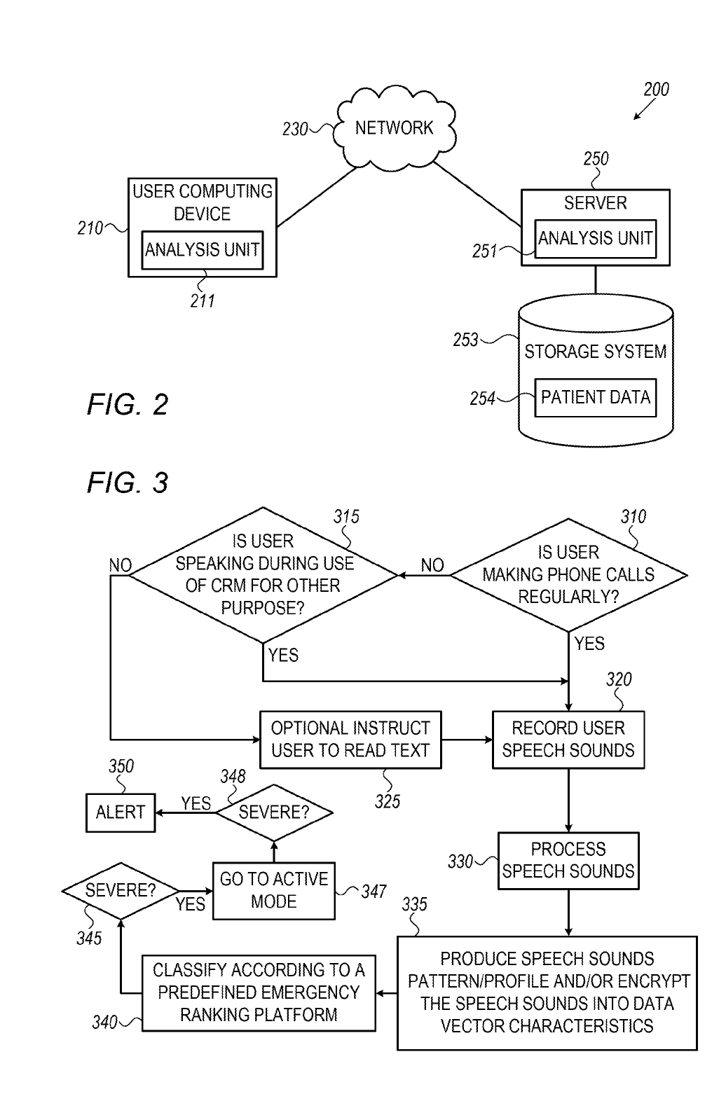

A system according to some embodiments of the invention may include components such as, but not limited to, a plurality of central processing units (CPU) or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, a tablet, a kiosk, a smartphone, a telephone, a smart watch, a wearable device, a medical device and any combination thereof, or any other suitable computing device. For example, a system as described herein may include one or more devices such as computing device 100.

Where applicable, units shown by FIG. 2 and other components and units described herein, may be similar to, or may include components of, device 100 described herein. For example, server 250 shown in FIG. 2 and further described herein may be or may include a controller 105, memory 120 and executable code 125. More than one computing device 100 may be included in a system, and one or more computing devices 100 may act as the various components of a system, for example, the components of system 200 such as user computing device (UCD) 210 and server 210 shown in FIG. 2.

As described, the present invention enables a home tele-monitoring system based on an algorithm designed to diagnose and/or monitor diseases such as LD, CHF, CKD and/or other diseases that affect speech such as depression, neurological diseases, psychiatric diseases and the like. As described, a system may record user speech sounds and define or create a personalized speech pattern or profile of the user.

An embodiment may provide an alert and feedback regarding a patient's condition and/or any development or progress in his physical and medical status to one or more of: the user, a physician, a healthcare systems and/or any medical staff or institute connected to the system. An embodiment may record patient's (or user's) speech sounds on different time intervals, e.g., periodically or whenever a device is used, e.g., for a telephone conversation. In some embodiments, a matrix or vector of parameters or values may be created by analyzing user speech sounds and data obtained by other sensors. An algorithm or logic used for analyzing user speech sounds may be employed, e.g., by a user computing device and/or be a server.

An embodiment may analyze speech sound patterns with respect to a profile, e.g., an embodiment may compare speech sound patterns of a patient to speech sound patterns obtained when the patient is in a reference, healthy state, accordingly, an embodiment may determine, identify and indicate a medical status of the patient. An embodiment may perform a dimensionality reduction of user speech sounds or characteristics, e.g., a set of values calculated based on analysis of speech of a user may be encrypted and included in a data vector. A vector as referred to herein may be a set, array, or sequence of values of a respective set, array, or sequence of parameters, e.g., a vector may be a set, array, or sequence of values of a frequency, amplitude and the like.

In some embodiments, a ranking scale or platform may be used in order to rank, score, or otherwise quantify a severity of a disease. For example, analysis unit 211 and/or analysis unit 251 may rank or score a state of a disease (or otherwise determine a severity of the disease) based on rules, thresholds and criteria included in a ranking data 132 as described.

Determining a medical condition, e.g., identifying or determining a presence, state or progress of a disease of a user, may include or may be based on a score or rank that may be associated with, attributed to, or calculated for, a presence, state or progress of a disease of a user.

For example, even if the speech characteristics of two patients are similar or same, different medical conditions, severities or scores may be calculated or determined for the two patients, e.g., based on their patient data 254, e.g., two users may have similar speech characteristics, but a high score or severity may be calculated for the first user who suffers from asthma and a low score or severity may be determined, calculated or set for the second user who does not suffer from asthma. Any rule or threshold may be included, e.g., in ranking data 133, such that any data or information included in patient data 254 may be taken into account when calculating a score, severity, trend, improvement, deterioration or other aspects of a uses medical condition as described herein. In some embodiments, if a score or rank calculated as described is above a threshold score or value then an action may be performed, e.g., if a score calculated by analysis unit 211 as described is above a threshold then analysis unit 211 may generate an alarm, alert the user and/or a physician, send a message to server 250 or perform any action as described herein.

A score or rank, e.g., a severity score may be calculated, e.g., based on comparing recorded speech 132 to user speech profile 131 (e.g., based on rules or thresholds in ranking data 133 as described) a severity score may be calculated. For example, a first severity score may be calculated if one speech characteristics value is above or below a threshold, a second, higher severity score may be determined if two speech characteristics are above or below a respective two thresholds and so on. Of course, a severity score may be based on the magnitude of a breach of a threshold, e.g., a first severity score may be determined if a pitch increases by 10% with respect to a previously measured pitch or with respect to a pitch in user speech profile 131 and a second, higher severity score may be determined if the pitch increases by 25%. A severity score may be sent to a server (e.g., analysis unit 211 may send a severity score to analysis unit 251) and, an alert or alarm may be generated and sent or provided if the severity score is above a predefined threshold, e.g., analysis unit 251 may send an alert message as described.

In some embodiments, based on comparing a speech characteristics vector of a user to a reference speech characteristics vector or to a user speech profile, a severity or score may be determined for, or associated with, a medical condition of the user. An action may be performed based on the severity or score, e.g., a first severity or score may cause an embodiment to generate an alarm, a second (e.g., lower) severity or score may cause an embodiment to take another measure (or recording) of user's speech and so on.

A ranking scale or platform may be used in order to rank, score or determine a severity of, a medical condition of a user. For example, analysis unit 211 and/or analysis unit 251 may rank, score or determine a severity based on rules and criteria included in a ranking definition (e.g., in ranking data 133) as described.

Any rule may be included, e.g., in ranking data 133, such that any data or information included in patient data 254 may be taken into account when calculating a score, severity, trend, improvement, deterioration or other aspects of a uses medical condition as described herein.

A set of thresholds, rules and/or criteria, e.g., included in ranking 133 may be used in order to determine a medical condition, e.g., in order to determine a state, progress or presence of a disease or a trend and/or an improvement or deterioration of a medical condition. For example, ranking data 133 may indicated that, if the length of pauses between words as determined or calculated based on recorded speech 132 is greater than the length of pauses as included or indicated in user speech profile 131 by more than 6 or by more than 20% than a deterioration or worsening of a disease is identified.

Any rule, criterion or threshold may be included in ranking data 133. For example, complex rules in ranking data 133 may include a breach of a number of thresholds related to a number of speech characteristics. For example, a critical condition may be identified, by analysis unit 211 if a pitch increases by 15% and the length of pauses between words by increases by 10%. Thresholds, rules and/or criteria in ranking data 133 may be based on any information related to a user. For example, a first set of thresholds, rules and/or criteria may be used for a child, a second set of thresholds, rules and/or criteria may be used for an adult, a third set may be used for an elderly female and so on. Thresholds, rules and/or criteria in ranking data 133 may be automatically and/or dynamically modified. For example, if or when an alarming condition is identified as described, analysis unit 211 may automatically modify thresholds, rules and/or criteria in ranking data 133 such that values or changes in speech characteristics that were previously regarded as normal (or of low severity) may now be regarded as indicating cause for alarm or high severity. For example, ranking data 133 may modified by analysis unit 211 or it may be downloaded, from or by, server 250, each time a change in the user's medical condition is identified or made known to system 200. For example, results from an ultrasound or other scan of a user may be provided to server 250 and, based on the results, ranking data 133 may be modified such that speech characteristics previously regarded as normal may now be regarded as abnormal or indicating a severity above a threshold. Other causes for automatically modifying thresholds and rules in ranking data 133 may be a new prescription, new symptoms and/or any information relevant to a medical condition of a user.

Ranking of a speech characteristics vector and/or determining that a threshold was breached may be based on patient data 254. For example, a deviation of 0.8 in the average pause between words may be treated as cause for alarm if, as indicated patient data 254, the user is 80 years old and suffers from a known disease (e.g., OLD) in but may be regarded as normal (and therefore may not cause an embodiment to generate an alarm) if the patient or user is 45 years old.

A database (e.g., in storage system 253 as described herein) may include personal medical file of a user, medical history, current medical data, allergies, chronic and medical treatment, e.g., patient data 254 described herein may include any medical and/or demographic data of a patient. For example, patient data 254 may include a physical symptom, physiological data, physical data, and use of medications. A server (e.g., server 250) may use data in a data base in order to analyze speech sounds or audio signals captured by a computing device as described and deduce, identify or determine a presence, state or progress of a disease of a user. Determining a presence, state or progress of a disease of a user may include identifying, detecting or determining any relevant aspect of a disease, for example, determining or identifying, by an embodiment, a presence of a disease may include identifying or detecting that a healthy user, e.g., one with no known medical history of a disease is now showing symptoms that may indicate the user has the disease, in other cases, determining or identifying, by an embodiment, a state, trend or progress of a disease may include identifying an improvement or worsening of, or related to, a disease.

Identifying or determining a state, trend or progress of a disease may include identifying or determining and quantifying a rate of change. For example, based on repeatedly comparing audio signals to a reference audio signal as described (e.g., over a number of days or weeks) the rate of improvement (or deterioration) of a disease may be determined and quantified. For example, ranks and scores calculated as described herein (e.g., based on comparing a recently captured audio signal to a reference audio signal) may be recorded over time and, based on a set of scores (e.g., calculated over days or weeks), the rate of change of a medical condition may be determined For example, based on a set of scores or ranks, an embodiment may determine how fast a disease is deteriorating or improving, accordingly, an effect or efficiency of a treatment may be measured or quantified. For example, after prescribing a specific medicine or other treatment for a disease, a physician may review reports from an embodiment that show or indicate a trend (e.g., an improvement or deterioration of the disease) and moreover, based on reports from a system (e.g., reports from server 250) that may include a rate of change as described, the physician may see or conclude the efficiency of the treatment based on the rate with which the patient is improving.

Any information related to a medical condition of a user or patient may be included in patient data 254 and may be used for determining a state or progress of a disease of the user. For example, thresholds used for identifying a state or progress of a disease, e.g., a deterioration of a medical condition or disease of a user may be set according to, or based on, medical information in patient data 254. For example, patient data 254 may include known (e.g., current, recent and/or historical) vital signs of a user (e.g., heart rate, blood pressure and the like), medications prescribed and/or used, symptoms the user has or suffers from, disease in the family, historical medical procedures or operations and the like. Thresholds used as described may be set or calculated based on patient data 254, for example, a first threshold for a minimal pitch (or for an average pause between spoken words) may be set for a first patient if patient data 254 indicates that the patient is using a specific medication or that a specific surgery or other procedure was performed and a second threshold for a minimal pitch may be set for a second patient if patient data 254 of the second patient indicates other medications or surgeries.

An embodiment may provide feedback or indication regarding a current condition of the patient to the patient, to a physician, to a health care institute or to a member of a medical stuff. For example, server 250 may send alert messages, over a communication network, to a list or recipients as described herein.

Reference is made to FIG. 1B that shows a user using an exemplary device 100 according to illustrative embodiments of the present invention. As shown, device 100 may be, or may be included in, a smartphone (e.g., device 100 as shown by FIG. 1B may include a processor 105 and a memory 120).

Accordingly, operations such as determining a state or progress of a disease of the user, detecting cough and/or breathing, generating and/or updating a reference audio signal may be performed in the background, while the user is using device 100 for various purposes (e.g., for playing games or for phone calls as shown by FIG. 1B), additionally, these operations may be performed without the user being aware that such operations are performed.

Reference is made to FIG. 2, an overview of a system 200 according to some embodiments of the present invention. As shown, a system 200 may include a UCD 210 that may include an analysis unit 211. Analysis unit 211 may be, or may include, a controller 105, a memory 120 and executable code 125 as described herein. As further shown, a system may include a server 250 that may include an analysis unit 251. Analysis unit 251 may be similar to analysis unit 211. As shown, server 250 may be operatively connected to a storage system 253 that may include or store patient data 254. As shown, a system 200 may include a network 230 that may enable server 250 and UCD 210 to communicate, e.g., exchange digital information as known in the art.

Network 230 may be, may comprise or may be part of a private or public IP network, or the internet, or a combination thereof. Additionally or alternatively, network 230 may be, comprise or be part of a global system for mobile communications (GSM) network. For example, network 230 may include or comprise an IP network such as the internet, a GSM related network and any equipment for bridging or otherwise connecting such networks as known in the art. In addition, network 230 may be, may comprise or be part of an integrated services digital network (ISDN), a public switched telephone network (PSTN), a public or private data network, a local area network (LAN), a metropolitan area network (MAN), a wide area network (WAN), a wireline or wireless network, a local, regional, or global communication network, a satellite communication network, a cellular communication network, any combination of the preceding and/or any other suitable communication means. Accordingly, numerous elements of network 230 are implied but not shown, e.g., access points, base stations, communication satellites, global positioning system (GPS) satellites, routers, telephone switches, etc. It will be recognized that embodiments of the invention are not limited by the nature of network 230.

In some embodiments, UCD 210 may include, or may be connected to any sensor or measuring device system or equipment adapted to capture or obtain information or data that may be used in order to determine a state or progress of a disease of the user. For example, I/O devices connected to UCD 210 may include a gyroscope, an accelerometer, a heart rate sensor, a temperature sensor, a GPS, a stethoscope, a nasal pressure transducer, a CO2 sensor, a mercury strain gauges, a respiratory inductance plethysmography, a Blood-Oxygen saturation (SpO2) sensor, a camera, a thermometer, an electrocardiography (ECG) sensing system, an Otoscope, a tongue depressor, a blood pressure monitor, a pulse oximetry, a Spirometer, a gas sensor, a pressure sensor, a chemical sensor. Server 250 may be connected to, or may obtain data from an Ultrasound system, a medical imaging system and the like. Accordingly, it will be understood that any medical information of a patient as known in the art may be available to a system and may be available and used when analyzing speech data as described herein.

Reference is made to FIG. 3, a flowchart of a method according to illustrative embodiments of the present invention. As shown by block 310, an embodiment may determine whether or not a user is regularly using a telephone or other communication device for making phone calls. For example, UCD 210 may be a smartphone as described and analysis unit 211 may be alerted, e.g., by an Android OS, each time a user is using the smartphone for a phone conversation or for recording messages. For example, using an application program interface (API) as known in the art, analysis unit 211 may be notified when a user is using a smartphone for talking.

As shown by block 315, an embodiment may determine whether or not a user (who is operating or using user device 210) is speaking, e.g., when or while using UCD 210 for any purpose. For example, UCD 210 may be a laptop and, while the user is surfing the internet, the user may be talking, e.g., with a friend in the same room. For example, determining whether or not a user is speaking may be achieved by repeatedly or periodically, or based on an event, activating a microphone in UCD 210, recording speech (or audio signals) and comparing recorded speech (or audio signals) to speech, or audio signals recording of the user. For example, user speech profile 131 (or recorded speech 132) may include a recording of the user's audio signals or speech and, accordingly, by comparing input from a microphone to user speech profile 131 and/or recorded speech 132, analysis unit 211 may determine whether or not a specific user is speaking For example, in some embodiments, each time a user is speaking, analysis unit 211 may identify or determine that the user is speaking as described and, if it is determined that the user is speaking, analysis unit 211 may record the user's speech. By identifying the user (e.g., based on recorded speech of the user as described) embodiments of the invention avoid the false alarms or other errors, e.g., if the owner of UCD 210 (a first user) gives his smartphone to another user (a second user) and the second user uses the smartphone for a phone conversation then although analysis unit 211 may be alerted as described, analysis unit 211 may identify or determine that the speech or audio signal picked up by the microphone of UCD 210 is not a speech of the first or owner of UCD 210 and may avoid recording or analyzing the speech as described.

As shown by block 325, an embodiment may prompt a user to speak, e.g., read presented text and may record user's speech. For example, if analysis unit 211 determines that user's speech has not be obtained or captured for more than a predefined period of time (e.g., one day) then analysis unit 211 may prompt the user to speak and may record user's speech as described. In some embodiments, e.g., in order to create a baseline profile, analysis unit 211 may instruct a user to read text and further provide guidance, e.g., request the user to read slower or faster, louder or software and the like, analysis unit 211 may record user's speech and guide the user as described until a clear, good quality audio signal of the user's speech is captured. As shown by block 320, user speech may be recorded, e.g., audio signals picked up by a microphone of UCD 210 may be stored in storage system 130 as shown by recorded speech 132.

Any method for causing a user to speak or produce sound or audio signals that may be captured and used as described may be used. For example, in order to record use's speech, an embodiment (e.g., an application on a smartphone) may instruct a user or patient to count from 1 to 10 or the application may ask the user a question (e.g., present the question as text on a display or use a speaker of computing device 100). For example, using a synthesized or other voice, an application may ask the user “how was your day?” or “how is the weather today?” and the application may record the users answer.

For example, if a difference between a recorded audio signal of the user and a baseline or reference audio signal is greater than a threshold then analysis unit 211 may revert to an active mode of determining a medical condition, for example, an active mode may include instructing the user to speak, recording the user's speech and determining a progress of a disease based on comparing the recorded speech to a baseline audio signal. For example, conditions that may cause a large difference as described may be, or may include, an exacerbation of a disease, someone other the user or owner of UCD 210 used UCD 210, a recent physical activity of the user, or an error. According to some embodiments, the active mode may further or alternatively include one or more of: recording the user's breathing; and prompting the user to input user's symptoms e.g. by presenting to the user a questionnaire on a display of UCD 210 and asking the user to answer questions. The active mode may further include determining the progress of the disease based on recorded breathing of the user and received symptoms.

In some embodiments, e.g., when operating in active mode as described, an embodiment may prompt a user to provide information that may be used in order to better determine a medical condition of the user. For example, by presenting text on a screen of UCD 210, by using text to speech or by playing recorded questions, analysis unit 211 may ask the user whether or not he or she just ran, did someone else use his or her device etc. Analysis unit 211 may ask the user any question, e.g., about coughing, sputum, sputum color, sputum amount, breathing difficulty, wheezing, general feeling, medications and so on. Based on information provided by a user as described, analysis unit 211 may modify thresholds and/or perform actions. For example, based on input from a user received in response to questions as described, analysis unit 211 may change thresholds and/or criteria in ranking data 133 and/or may perform a flow as described with reference to an active mode, e.g., prompt the user to speak, record speech and evaluate a state of a disease as described.

By reverting to active mode as described and repeating an assessment, an embodiment may verify that a severe medical condition (e.g., a deterioration of a disease), if identified, was correctly identified and accurately reflects or indicates the patient condition. Accordingly, accurate diagnosis of a patient's condition may be achieved by some embodiments as well as reduced cases of errors or false positives as known in the art.

As shown by block 330, speech may be analyzed, and, as shown by block 335, speech sound patterns, a profile and/or a speech characteristics vector may be produced, generated or created based on analysis of user speech. For example, a profile or a speech characteristics vector may include values or representations of speech, audio or acoustics parameters, e.g., values or representations of an amplitude or power, an energy level, a frequency, a maximum and minimum pitch, a length of pauses, energy differences over frequency bands and the like.

A speech profile (e.g., user speech profile 131) may include a specific set of values for a predefined set of parameters as derived or determined based on speech of a user. A speech profile (e.g., user speech profile 131) may be created based on normal speech of the user, e.g., when the state of a disease and/or the medical condition of the user is known, and, accordingly, user speech profile 131 may represent the user and may be used in order to assess or determine the state or progress of a disease of the user described herein. A speech characteristics vector may be, or may include values or representations of speech, audio or acoustics parameters, e.g., as described with respect to user speech profile 131. For example, user speech profile 131 may describe or represent a user as described and a speech characteristics vector may be a temporal, realtime or current representation of the user. It will be understood that any number of profiles or user profiles may be used and that user speech profile 131 is an exemplary profile. For example, user speech profile 131 or an additional profile may include characteristics of specific sounds or audio signals of a user. For example, a profile may include characteristics of coughs and/or sneezes (e.g., dominant or other audio frequencies, amplitudes and the like) such that specific sounds may be characterized and/or such that a reference for specific sounds may be created. Accordingly, an embodiment may determine a state or progress of a disease of a user based on identifying specific sounds, creating a reference for specific sounds and comparing the specific sounds, as captured from the user, to a reference.

As shown by block 340, a vector or other data produced as described (e.g., with reference to block 335) may be classified according to a predefined ranking or classification definition, algorithm or any rule, threshold or criterion. For example, ranking or classification definition may be, or may include a threshold for a minimal pitch, e.g., a decrease of more than 20% of a maximal observed pitch indicates a deterioration that requires alarming the user and/or a physician. Another rule, threshold or criterion may be related to pauses, e.g., analysis unit 211 may compare an average pause between spoken words, as determined based on a recently captured speech of the user to the average pause between spoken words as included in user speech profile 131 and, if the current or recent average pause is 0.8 or less of the average pause in user speech profile 131 then analysis unit 211 may perform at least one action.

In some embodiments, if a deterioration in the medical condition of a patient (e.g., a worsening of a disease) is detected or suspected, an embodiment may interact with the, e.g., using the patient's smartphone, an embodiment may instruct the patient to lie down and rest, call an ambulance or perform any other operation. In order to verify that the medical condition of a patient is indeed deteriorating or is such that an alarm should be generated as described, an embodiment may instruct a patient to speak (e.g., as described herein) and may record audio signals as described herein, e.g., in order to reevaluate the patient's medical condition and determine whether or not an identification or determination of a deterioration in medical condition is erroneous (e.g., a false positive as known in the art). Accordingly, based on an identified medical condition (e.g., based on identifying a state or progress of a disease), an embodiment may interact with a patient and provide the patient with instructions designed to best handle a medical emergency and/or acquire additional medical readings or measurements of the patient.

In some embodiments, as shown in block 347, if, based on comparing a sound characteristics vector representing a current condition of a user to a user speech profile 131 analysis unit 211 determines or identifies a deviation of one or more characteristics is above a threshold (e.g. severe change) then analysis unit 211 may go into active mode and prompt the user to speak, e.g., while sitting up straight or lying down, may capture user's speech as described, create (or recreate) a speech characteristics vector and verify that conditions for alarm indeed exist by comparing the newly created speech characteristics vector to the user's speech profile (or to a reference speech characteristics vector), and may recheck the deviation of the one or more characteristics and if such deviation is severe (as indicated in block 348) issue an alarm. Accordingly, false alarms may be avoided or reduced significantly. According to some embodiments, the active mode may further or alternatively include one or more of: recording the user's breathing; and prompting the user to input user's symptoms e.g. by presenting to the user a questionnaire on a display of UCD 210 and asking the user to answer questions. The active mode may further include determining the progress of the disease based on recorded breathing of the user and received symptoms.

Analysis unit 211 may perform any action based on a classification or examination of recorded speech. For example, based on comparing a speech characteristics vector representing a current condition of a user to a user speech profile 131 that represents a normal (or known) condition of the user and determining a breach of at least one threshold, analysis unit 211 may: send a message to a predefined list of recipients (e.g., a physician of the patient, a medical institution, a family relative and the like); sound an alarm (e.g., using a speaker in UCD 210), present a warning message (e.g., using a display of UCD 210) or perform any other action. In some embodiments, if, based on comparing a speech characteristics vector representing a current condition of a user to a user speech profile 131 analysis unit 211 determines or identifies a deviation of one or more characteristics is above a threshold then analysis unit 211 may prompt the user to read text, capture user's speech as described, create (or recreate) a speech characteristics vector and verify that conditions for alarm indeed exist by comparing the newly created speech characteristics vector to the user's speech profile, accordingly, false alarms may be avoided.

As shown by block 345, a speech characteristics vector created as described may be sent or uploaded to a server, e.g., analysis unit 211 may send or upload a speech characteristics vector to server 250 where analysis unit 251 may analyze the uploaded speech characteristics vector, e.g., as described with reference to analysis unit 211. As shown by block 350, an alert may be generated and/or sent, e.g., by server 250. For example, an alert generated, presented or sent by server 250 may be or may include an electronic mail sent to one or more recipients, an audible or visual alarm and the like.

Server 250 may analyze, examine or process a speech characteristics vector based on any data or information related to a user or patient, e.g., data included in patient data 254. For example, patient data 254 may include medical history and condition and/or demographic information of a user of UCD 210 and server 250 may analyze a received speech characteristics vector based on such data. Ranking of a speech characteristics vector and/or determining that a threshold was breached may be based on patient data 254. For example, a deviation of 0.8 in average pause may be treated as cause for alarm if the user is 80 years old and suffers from a known disease (e.g., CHF or CKD) but may be regarded as normal if the patient or user is 45 years old.

As described, an embodiment (e.g., controller 105 included in analysis unit 211) may receive an audio signal related to a user's speech and may determine a state or progress of a disease of the user based on comparing the audio signal to a reference audio signal. For example, UCD 210 may be a mobile communication device (e.g., a smartphone) owned and/or operated by a user or patient and, when a user uses UCD 210 for a phone call, analysis unit 211 may automatically record the user's speech or audio signal. By automatically, in the background and without user intervention, awareness or effort, obtaining user's speech sounds as described, processing the obtained speech as described, determining a state or progress of a disease and, if need be, alerting as described, a system and method may provide or enable advantages over known or existing systems and methods. The advantages of an automated system and method that continuously, periodically and/or repeatedly monitors and determines a user's medical condition (e.g., monitors a state or progress of a disease) without burdening the user or patient and/or a medical professional may be appreciated by a person skilled in the art.

As described, a progress of a medical condition (e.g., a state or progress of a disease), for example, a deterioration or improvement of symptoms or effects of a disease, may be determined based on comparing an audio signal generated by a user with a reference audio signal. As described, a reference audio signal or speech of a user may be a recording of the user captured or taken during normal speech of the user. For example, after ascertaining the user is in good or normal condition, analysis unit 211 may prompt the user to read text displayed on a display of UCD 210 and may record the user's speech. Speech recorded while, or during a time period, the user is in good or known condition may be used for generating a reference audio signal or a reference speech characteristics vector. Once a reference speech characteristics vector or reference speech recording is created and stored, e.g., in storage system 130 and/or in storage system 254, an embodiment may use it in order to continuously, periodically and/or repeatedly determine a trend or progress of a user's medical condition (e.g., determine a state or progress of a disease) by continuously, periodically and/or repeatedly capturing the user's speech sounds (e.g., every time the user speaks near UCD 210) and compare captured user's speech sounds or audio signals generated by the user to the reference speech characteristics vector or reference speech recording. For example, for each new recording of user's speech, analysis unit 211 may generate a speech characteristics vector by extracting, from the recording, values such as amplitudes, frequencies, pitch levels and the like, include the extracted values in a speech characteristics vector and compare the speech characteristics vector to the reference speech characteristics vector.

In some embodiments, determining a user's medical condition and/or a trend, state or progress a disease may be based on, or according to, any audio signal produced by the user. For example, an embodiment may capture or identify the cough or sneezing sounds of a user, record frequency of coughs or sneezes and, if the frequency of coughs or sneezes (e.g., number of coughs or sneezes per day, hour or minute) increases above a threshold then an embodiment may determine that the medical condition of the user is deteriorating. Audio analysis of specific sounds (e.g., audio analysis designed to identify or detect coughs or sneezes) may be performed and characteristics of specific sounds may be recorded (e.g., in a user profile such as user speech profile 131 or in another profile as described) such that a reference or baseline for specific sounds may be created and used as described. For example, a reference or baseline may indicate or include the number of coughs or sneezes per minute or hour, the audio frequencies of the coughs or sneezes etc., and specific sounds captured as described may be compared to the reference or baseline of specific sounds in order to identify or determine trends or condition as described.

In some embodiments, identifying or detecting coughs or sneezes may be performed without comparing a recorded speech to a profile or baseline. For example, audio analysis used by analysis unit 211 may enable analysis unit 211 to identify a cough or sneeze based on frequency or amplitude variations as known in the art. Accordingly, an embodiment may identify or detect an illness even without a profile as described. For example, an embodiment may record or capture user's speech whenever the user speaks, use speech or audio analysis to search, identify, or look for, specific phenomena (e.g., hoarseness, cough, sneeze and the like) and, if analysis unit 211 identifies or detects a specific phenomenon, user analysis may perform an action as described, e.g., alert the user, alert a physician and so on.

Comparing vectors may be done as known in the art. For example, relating or comparing vectors (e.g., comparing a speech characteristics vector to a reference speech characteristics vector as described) may include defining and using a space of interest. Otherwise described, relating or comparing vectors may include relating or comparing the projections of the vectors on a predefined space.

For example, distance between a speech characteristics vector and a reference speech characteristics vector in a predefined space (e.g., a space defined by coordinates that are a frequency and amplitude) may be calculated or determined as known in the art and the distance may be compared to a threshold or predefined value. In some embodiments, if the distance between a speech characteristics vector and a reference speech characteristics vector is above a threshold, the embodiment may perform an action as described, e.g., generate and send an alarm message, display a warning on a display of UCD 210, send an email to a physician and so on.

It will be understood that any space of any dimension for comparing vectors may be defined and, accordingly, that vectors of any dimension may be compared or matched. Accordingly, the dimensionality or space used for vector operations may be reduced. For example, if it is known that LD or progress thereof may be identified based on two aspects of user's speech (e.g., frequency and amplitude) then an embodiment may use a two-dimension vectors and space order to evaluate, determine or assess a condition or progress of LD of a user.

As described, recorded audio signals or speech may be uploaded to a server (e.g., to server 250) and the server may determine a state or progress of a disease of the user based on comparing the audio signal to a reference audio signal and based on any medical or other information related to the user. For example, examining or comparing vectors as described may be based on a user's medical record or demographic data. For example, as described, an embodiment may generate an alarm if a distance between a speech characteristics vector and a reference speech characteristics vector is greater than a threshold. In some embodiments, the threshold may be user specific, and/or dynamically set. For example, in some embodiments, a first threshold related to vector distance as described may be used for a patient who suffers from LD, a second threshold may be used for a patient who suffers from COPD, a third threshold may be used for a patient with Asthma and so on.

As described, embodiments of the invention improve the field of monitoring patients, e.g., by enabling a system and method that continuously, periodically or repeatedly monitors and determines a presence, state or progress of a disease without requiring dedicated equipment to be attached to a patient and further without burdening the patient.

Embodiments of the invention address the computer-centric challenge of computerized monitoring related to health or medical condition. Unlike known systems and methods that require and use dedicated devices or systems, embodiments of the invention address the computer-centric challenge of computerized health monitoring and alerting using a device that is normally carried and operated by a user (e.g., using a smartphone as described).

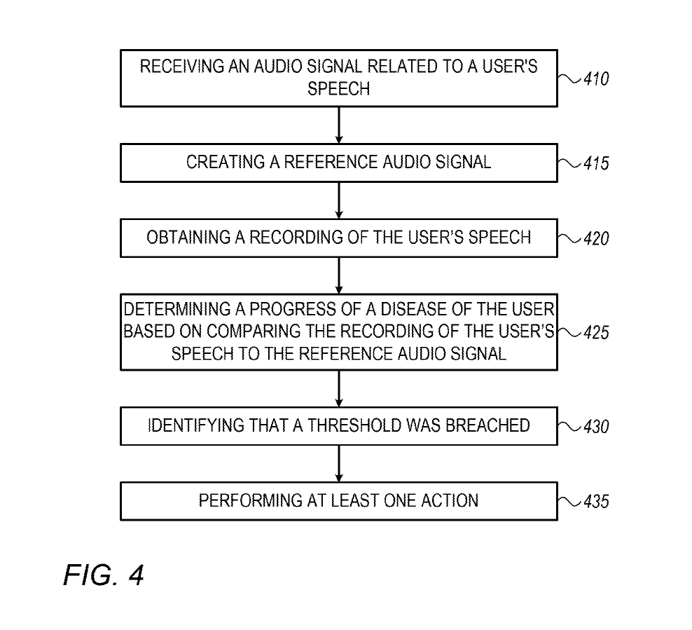

Reference is made to FIG. 4, a flowchart of a method according to illustrative embodiments of the present invention. As shown by block 410, an audio signal related to a user's speech may be received. For example, analysis unit 211 may receive, from a microphone included in UCD 210, an audio signal as described. As shown by block 415, a reference audio signal may be created. For example, analysis unit 211 may create a reference audio signal based on recording normal speech of the user as described. A reference audio signal may be continuously, periodically or iteratively updated, e.g., by periodically recording speech of the user during telephone calls made by the user and updating the reference audio signal, for example, recorded speech 132 may be used in order to update user speech profile 131 such that user speech profile 131 is kept up-to-date and reflects the user's current state, e.g., reflects or represents the user's medical condition in terms of speech sounds or characteristics. In some embodiments, a reference audio signal may be created by presenting text to a user, instructing the user to read the text, recording the user's speech while the user reads the text, and creating the reference audio signal based on the recording and/or based on speech sounds or characteristics extracted from the recording. For example, a reference audio signal may include sounds or characteristics such as pitch, amplitude and the like. Other methods may be used, e.g., requesting the user to count, asking the user a question and the like.

As shown by block 420, a recording of the user's speech may be obtained. For example, after a reference audio signal (or a baseline as described herein) is created, an embodiment may record the user when he or she speaks. As shown by block 425, a progress of a disease may be determined based on comparing the recording of the user's speech to the reference audio signal. For example, analysis unit 211 may compare a new or recent recording of speech of a user to a reference audio signal (e.g., included in user speech profile 131) and determine a presence, state or progress of a disease based on differences found between the newly or recently recorded speech and a reference audio signal, a baseline or a profile of the user as described. As shown, a breach of a threshold may be identified (also referred to as anomaly detection or outlier detection). For example, a breach of one or more thresholds related to variations or differences of speech, sound or audio characteristics, may be determined or identified by comparing the characteristics in a recorded speech to the characteristics in a profile (e.g., user speech profile 131) and/or to the characteristics in a baseline or in a reference audio signal, e.g., by analysis unit 211 as described.

As shown by block 435, at least one action may be performed. For example, if analysis unit 211 identifies that a threshold was breached or a calculated score is greater than a predefined value then analysis unit 211 may generate an alarm or alert as described.

In some embodiments, thresholds may be dynamically modified based on various inputs, aspects or considerations. For example, thresholds may be modified based on user associated parameters (such as location, medical history, recent hospitalizations, etc.) weather parameters (temperature, humidity, etc.), and the like. According to some embodiments, the threshold of each patient or user may be personalized based on anomaly detection. For example, a threshold included in ranking data 133 as described may be defined, set or updated based on a location of the user or the location of UCD 210. For example, analysis unit may receive a location of UCD 210 from a GPS unit included in UCD 210 and may alter or modify a threshold based on the location.

In some embodiment, determining a location of a user may be based on input received from a component included in a system. For example, analysis unit 211 may determine a location of a user based on input received from a GPS unit included in UCD 210, e.g., a GPS unit included in a smartphone as known in the art. Any system or method for determining a location of a user may be used. For example, a location of a user may be determined based on known or identified nearby WiFi access points (e.g. using the Wi-Fi positioning system (WPS) as known in the art, or the GLONASS system may be used or the location may be provided by a user.

For example, as known in the art, speech or sound characteristics (e.g., pitch) may vary based on altitude, pollution, weather and the like. Accordingly, if it is determined, based on a location, that the user is at high altitude, analysis unit 211 may change a threshold related to pitch such that the variation of the user's speech caused by the high altitude is taken into account, e.g., the threshold value may be increased to accommodate a shift in pitch that may be caused by thinner air in high altitudes.

In embodiments, analysis unit 211 may report a location of UCD 210 to server 250 and receive, from server 250, information relevant to the location, e.g., weather conditions, level of pollution, dust or smoke levels in the area and the like. As described with reference to altitude, analysis unit 211 may dynamically modify thresholds, criteria or logic, e.g., included in ranking data 133 such that aspects such as weather conditions, level of pollution and the like are taken into account when determining a progress or severity of a disease as described.

In some embodiments, a threshold (e.g., included in ranking data 133 as described) may be defined, set or updated based on an activity of the user. For example, as known in the art, a person's speech (or sound characteristics) may vary when physically strained.

In some embodiments, analysis unit 211 may determine or identify an activity of a user, e.g., based on input from an accelerometer unit (or a gyroscope unit) included in UCD 210, for example, analysis unit 211 may identify or determine that the user is now running or walking fast. Determining or identifying an activity of a user may be done or achieved using any known systems and methods. In some embodiment, identifying an activity of a user may be based on input received from a component included in a system. For example, analysis unit 211 may identify an activity of a user based on input received from an accelerometer or a gyroscope unit included in UCD 210, e.g., an accelerometer or a gyroscope unit included in a smartphone as known in the art. According to some embodiments, UCD 210 may measure the patient daily activity, e.g., how many steps, walking speed, hours of daily activity, heart rate during activity and other physiological measurements, such as number of breaths per minute. Any change in the measured daily activity, may be related to, or may indicate a worsening in the patient's disease.

Other components or methods may be used for identifying an activity, for example, data received from a GPS (e.g., one built-in UCD 210) may enable analysis unit 211 to determine the speed or velocity of the user, e.g., identify the user is running, input from an ECG unit indicating increased heart rate and/or any vital signs obtained as known in the art may indicate, and used for, determining physical activity.

Based on an activity of the user, analysis unit 211 may dynamically modify or change thresholds, for example, if it is determined that the user is running then thresholds related to pitch, pauses and/or frequency may be changed in order to accommodate natural changes or shifts in speech or sound characteristics.

In some embodiments, if analysis unit 211 determines whether or not the user of UCD 210 performed a physical activity prior to the determining of the progress of a disease and if the user performed a physical activity (possibly immediately) prior to the determining of the progress of a disease as described then analysis unit 211 may instruct the user to rest or lie down or sit. Either after a user confirms performing an instruction such as resting or after a predefined interval has passed since the physical activity was performed, analysis unit 211 may instruct, request or otherwise cause, the user to speak or produce sound (e.g., as described herein) and may reevaluate the user's medical condition, e.g., determine a state or progress of a disease as described herein. Accordingly, false positives as known in the art may be avoided.

An embodiment may determine nonadherence with a prescribed treatment based on at least one of: comparing the audio signal to a reference audio signal and a report from an adherence system. For example, analysis unit 211 may receive reports from a system that records or measures dosages of medicine (e.g., a system that tracks medication use as known in the art) and, based on the reports, determine whether or not the user is taking his or her pills at the right times and dosages, uses an inhaler and so on. Other devices or systems that may be operatively connected (e.g., over a Bluetooth, WiFi or other network) to analysis unit 211 may be an oxygen saturation metering system, a peak flow meter, a spirometer and so on.

If determining nonadherence as described, analysis unit 211 may remind the user to use medications (e.g., using a screen or speaker of UCD 210 as described). If determining nonadherence as described, analysis unit 211 may change thresholds or criteria such that nonadherence and its effect are taken into account when determining a state of a disease. For example, if it is known that a patient who suffers from a specific disease and who, in addition, is not taking his or her medicine, is at a higher risk then a threshold may be lowered such that an alarm that would not be generated when (or if) the user takes his or her medicine as prescribed will now be generated as described. For example, a rule may be specific to the user's medical condition and to the prescribed treatment (and possibly to a specific threshold) and, upon determining nonadherence as described, the rule may be used in order to modify the threshold.

Various methods may be used in order to identify or determine nonadherence. For example, if an improvement in medical condition of a user it is expected in light of a new prescription (that may be known to analysis unit 211, e.g., based on information received from server 250 or based on input from a user) but no improvement is identified during an indicated time period then analysis unit 211 may determine that the user does not adhere to the new prescription. For example, analysis unit 211 may receive a message from server 250 that, e.g., based on patient data 254, informs analysis unit 211 that the user is now expected to take a new pill and that an improvement is expected inside one week. In such case, if analysis unit 211 does not identify an improvement within a week, analysis unit 211 may determine nonadherence, e.g., determine that the user does not take his pills as prescribed.

For example, if medication A is taken 3 times a day, and changes are detected in speech patterns in the morning and the evening but not at noon, it may be assumed that the patient is skipping his noon dose.

Accordingly, an embodiment may accurately determine a medical condition and/or accurately identify a state or progress of an illness or disease even under changing and/or different conditions, in different locations, when medications are changed and so on. Moreover, false alarms (e.g., false positives) may be avoided by, when determining a state or progress of a disease or otherwise evaluating a medical condition as described, taking into consideration or account a location, an activity, an altitude, a weather and other aspects as described.

In some embodiments, a biomarker score may be calculated for a user by comparing an audio signal (recorded speech 132) to a reference audio signal, e.g., a reference audio signal that may be, or may be included in, user speech profile 131.

For example, by comparing recorded speech 132 to user speech profile 131 and/or by speech analysis as known in the art a biomarker score that indicates or quantifies speaking difficulty of the user may be produced. In other embodiments, a biomarker score may be used for evaluation in an emergency department or in a clinic visit, for monitoring progress during hospitalization or clinical trials.

Generally, a biomarker as known in the art may be any parameter that may indicate a medical condition. For example, a biomarker may be any detectable or measurable aspect or value, e.g., a symptom or a substance (e.g., in blood) that is indicative of phenomenon such as disease, exposure to toxic material, infection and the like. Known systems and methods use biomarkers such as readings or output of a spirometer, blood tests and the like. In some embodiments, a score or rank as described herein may be used as a biomarker. For example, when a new drug is being tested on COPD patients, in order to measure or validate the effectiveness of the new drug, a score or rank as described herein may be used, or treated as a biomarker that can indicate the effectiveness or effect of the drug, e.g., if the score (or severity score or rank) calculated for a patient as described herein decreases over time, this may indicate that the new drug is effective. The rate with which a score decreases may be used as a measure of effectiveness. For example, if two different new drugs are tested or evaluated (e.g., on two respective groups of patients) then the trends of scores (e.g., the amount or rate of decrease of severity scores) may be used in order to identify or determine which of the two new drugs is best. In another example, a score or rank as described herein may be used, or treated as a biomarker that can indicate changes in a patient status (e.g. changes in medical condition) during hospitalization. A baseline biomarker may be measured when the patient is admitted to the hospital (e.g. when the patient is received at the emergency room) and thereafter the biomarker may be repeatedly measured and compared to the baseline biomarker or to a previously obtained biomarker in order to determine the changes in the patient status during hospitalization and determine the effectiveness of the treatment given to the patient etc. It should be appreciated that using a score or rank as a biomarker as described above may assist in tracking improvement or decline in a patient's medical condition.

In some embodiments, analysis unit 211 may record the amount of speech of the user per time unit over a predefined time interval. For example, the number of spoken words per hour, minute or day may be recorded over any period of time (e.g., days or even months) or the time the user is actually speaking, singing or producing any other vocal signals may be recorded such that an embodiment may record, and provide if requested to, any statistics related to speaking or producing vocal signals. Analysis unit 211 may examine trends related to amount of speech over time and/or use thresholds as described in order to identify phenomena that require attention. For example, using data collected as described and a threshold as described, analysis unit 211 may identify a sharp decrease in speech (e.g., a user who use to speak normally now hardly speaks) and, based on such identification, may generate an alert. In other embodiments, identifying that a user speaks at specific hours and/or without being responded to by a party to a conversation, analysis unit 211 may identify a user talks in his or her sleep and may alert of such case.

In some embodiments, a user may be classified according to various features and/or statistics. For example, analysis unit 211 may record statistical data such as frequencies of features, e.g., amount or frequency of speech (e.g., number of spoken words or syllables per minute or between detected breathings), frequency (e.g., number per minute) of coughs, sneezes, breaths and so on. A user may be classified based on the statistical data or features as described herein. In some embodiments, a classification of a user may be provided, e.g., to the user himself or to an application. A classification may be used in various ways, for example, a research may use a classification as described in order to identify a relation between amount of speech and diseases, a research related to psychology may use the classification as described in order to identify relations between amount of speech and psychological aspects and so on.

In the description and claims of the present application, each of the verbs, “comprise” “include” and “have”, and conjugates thereof, are used to indicate that the object or objects of the verb are not necessarily a complete listing of components, elements or parts of the subject or subjects of the verb. Unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of an embodiment as described. In addition, the word “or” is considered to be the inclusive “or” rather than the exclusive or, and indicates at least one of, or any combination of items it conjoins.

Descriptions of embodiments of the invention in the present application are provided by way of example and are not intended to limit the scope of the invention. The described embodiments comprise different features, not all of which are required in all embodiments. Some embodiments utilize only some of the features or possible combinations of the features. Variations of embodiments of the invention that are described, and embodiments comprising different combinations of features noted in the described embodiments, will occur to a person having ordinary skill in the art. The scope of the invention is limited only by the claims.

Unless explicitly stated, the method embodiments described herein are not constrained to a particular order in time or chronological sequence. Additionally, some of the described method elements may be skipped, or they may be repeated, during a sequence of operations of a method.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A system comprising:
a memory; and
a processor included in a communication device, the processor configured to:
receive an audio signal related to a user's speech; and
determine a progress of a disease of the user based on comparing the audio signal to a reference audio signal.

2. (canceled)

3. The system of claim 1 wherein the reference audio signal is generated based on recording normal speech of the user.

4. (canceled)

5. The system of claim 1 wherein the processor is further configured to generate the reference audio signal by periodically recording speech of the user during telephone calls.

6. The system of claim 1 wherein the processor is further configured to obtain the audio signal by:
presenting text to the user;
instructing the user to read the text; and
recording the user's speech.

7. The system of claim 1 wherein the processor is further configured to communicate the audio signal to a server and wherein the server is configured to:
determine a progress of a disease of the user based on comparing the audio signal to a reference audio signal; and
based on the progress, send a message to a predefined recipient list

8. The system of claim 1 wherein the processor is further configured to alert the user based on the determined medical condition.

9. The system of claim 1 wherein determining a progress of the disease includes:
identifying, based on comparing the audio signal to a reference audio signal that a threshold is breached, wherein the threshold is defined based on at least one of: an activity of the user, a location of the user, medical history of the user, and recent hospitalization information of the user.

10-12. (canceled)

13. The system of claim 1 comprising:
determining whether or not the user performed a physical activity prior to the determining of the progress of a disease; and
if the user performed a physical activity prior to the determining of the progress of a disease then instructing the user to rest,
obtaining an audio signal related to a user's speech,
determining a progress of a disease of the user based on comparing the audio signal to the reference audio signal.

14. The system of claim 1 wherein the processor is further configured to:
if a difference between the recorded audio signal of the user and a baseline audio signal is greater than a threshold then the processor is configured to enter an active mode, wherein the active mode comprises:
instructing the user to speak;
recording the user's speech; and
determining a progress of the disease based on comparing the recorded speech to the baseline audio signal.

15-16. (canceled)

17. The system of claim 9 wherein the processor is further configured to:
determining nonadherence with a prescribed treatment based on at least one of: comparing the audio signal to a reference audio signal and a report from an adherence system; and
modifying the threshold according to a rule related to the user's medical condition and to the prescribed treatment.

18-19. (canceled)

20. A method of monitoring and determining a medical condition of a user comprising:
receiving, by a processor included in a communication device, an audio signal related to a user's speech; and
determining a progress of a disease of the user based on comparing the audio signal to a reference audio signal stored in a memory; and
performing at least one action based on the progress.

21. (canceled)

22. The method according to claim 20 wherein the reference audio signal is generated based on recording normal speech of the user.

23-24. (canceled)

25. The method according to claim 20 wherein obtaining the audio signal by the processor, further comprises:
presenting text to the user;
instructing the user to read the text; and
recording the user's speech.

26. The method according to claim 20 further comprising:
communicating, by the processor, the audio signal to a server;
determine, by the server, a progress of a disease of the user based on comparing the audio signal to a reference audio signal; and
based on the progress, sending by the server a message to a predefined recipient list.

27. (canceled)

28. The method according to claim 20 wherein determining a progress of the disease includes:
identifying, based on comparing the audio signal to a reference audio signal that a threshold is breached, wherein the threshold is defined based on at least one of: an activity of the user, a location of the user, medical history of the user, and recent hospitalization information of the user.

29-31. (canceled)

32. The method according to claim 20 further comprising:
determining whether or not the user performed a physical activity prior to the determining of the progress of a disease; and
if the user performed a physical activity prior to the determining of the progress of a disease then instructing the user to rest,
obtaining an audio signal related to a user's speech,
determining a progress of a disease of the user based on comparing the audio signal to the reference audio signal.

33. The method according to claim 20 further comprising:
if a difference between the recorded audio signal of the user and a baseline audio signal is greater than a threshold then enter an active mode, wherein the active mode comprises:
instructing the user to speak;
recording the user's speech; and
determining a progress of the disease based on comparing the recorded speech to the baseline audio signal.

34. The method according to claim 33 wherein the active mode further comprises one or more of: recording the user's breathing; and prompting the user to input user's symptoms; and wherein determining the progress of the disease is further based on recorded breathing of the user and received symptoms.

35. (canceled)

36. The method according to claim 28 further comprising:
determining, by the processor, nonadherence with a prescribed treatment based on at least one of: comparing the audio signal to a reference audio signal and a report from an adherence system; and
modifying, by the processor, the threshold according to a rule related to the user's medical condition and to the prescribed treatment.

37-38. (canceled)

39. A method of monitoring and determining a progress of a disease, the method comprising:
obtaining, by a processor included in a communication device, an audio signal of a user's speech; and
determining a progress of the disease by comparing the audio signal to a reference audio signal stored in a memory; and
based on the progress, selecting to perform at least one action.
